(19) United States
(12) Patent Application Publication
(10) Pub. No.: US 2003/0203366 A1
(43) Pub. Date: Oct. 30, 2003

(54) Microarray channel devices produced by a block mold process

(75) Inventors: Drahoslav Lim, San Diego, CA (US); Norman G. Anderson, Rockville, MD (US); James A. Braatz, Beltsville, MD (US)

Correspondence Address: John C. Robbins, Large Scale Biology Corporation, 3333 Vaca Valley Parkway, Suite 1000, Vacaville, CA, 95688, US

(73) Assignee: Large Scale Proteomics Corporation

(21) Appl. No.: 10/132,575

(22) Filed: Apr. 26, 2002

(51) Int. Cl. C12Q 1/68; C12M 1/34; G01N 33/543

(52) U.S. Cl. 435/6; 436/518; 435/287.2

(57) ABSTRACT

Microarrays are made from sections of a molded block having many channels. These channels, which are formed by casting and/or embedding a rod in a moldable solid, are used to immobilize biological and chemical binding components after rod removal. The microarrays can be used in general biological assays, clinical evaluations and chemical library analyses.

### FIELD OF THE INVENTION

The instant invention relates to microarrays containing bioreactive molecules, uses thereby and methods for manufacture thereof. Specifically, substrates or matrices are used to cast channels and/or simultaneously deposit bioreactive molecules onto molded inner surfaces of channels or voids within a block or mold presented by subsequent substrate or matrix purgation. The resulting molds or blocks contain unique reactants, where upon sectioning, large numbers of identical arrays are produced.

### BACKGROUND OF THE INVENTION

Synthesis and analysis of large numbers of bound oligonucleotides or peptides are generally known in the art. For example, the Selectide bead approach Kurka et al., Combinatorial Chemistry and High Throughput Screening 2(2):105-122 (April 1999) uses vast quantities of spherical cross-linked polymer beads (Millipore or Cambridge Research Laboratories polyacrylamide beads or Rapp Tentagel polystyrene) divided into 20 equal piles, each of which then has a different L-amino acid coupled to all the beads in a pile. The bead piles are then combined and thoroughly mixed. The resulting single pile is again divided into 20 different piles, each of which is reacted with a different one of the 20 different L-amino acids. This Divide, Couple and Recombine process (DCR) is repeated through six reactions to produce hexapeptides bound to the beads. Such sorting and resorting becomes too burdensome and labor intensive for the preparation of large arrays of peptides. Further, this process can be characterized as not calling for a continuous support, and it is not addressable.

Another approach, using arrays, is the pin dipping method for parallel oligonucleotide synthesis. Geysen, J. Org. Chem. 56, 6659 (1991). In this method, small amounts of solid support are fused to arrays of solenoid controlled polypropylene pins, which are subsequently dipped into trays of the appropriate reagents. The density of arrays, however, is limited, and the dipping procedure employed is cumbersome in practice.

Disclosed by Southern at Genome Mapping Sequence Conference, May 1991, Cold Spring Harbor, N.Y., (and U.S. Pat. No. 5,436,327) is a scheme for oligonucleotide array synthesis in which selected areas on a glass plate are physically masked and the desired chemical reaction is carried out on the unmasked portion of the plate. The problem with this method is that it is necessary to remove the old mask and apply a new one after each interaction.

Fodor et al., Science 251, 767 (1991) describe another method for synthesizing very dense 50 micron arrays of peptides (and potentially oligonucleotides) using mask-directed photochemical de-protection and synthetic intermediates. This method is limited by the slow rate of photochemical de-protection and by the susceptibility to side reactions (e.g., thymidine dimer formation) in oligonucleotide synthesis.

Khrapko et al., FEBS Letters 256, 118 (1989) suggest simplified synthesis and immobilization of multiple oligonucleotides and placing same by direct synthesis on a two-dimensional support, using a printer-like device capable of sampling each of the four nucleotides and placing same into given spots on the matrix. For example, the probes are applied to a chip with a pin or a pipette in the pattern of an array and immobilized by any of a variety of techniques such as adsorption or covalent linkage. An example of such DNA arrays is described in Stimpson et al. Proc. Natl. Acad. Sci. USA Vol. 92, pp. 6379-6383, July 1996.

Since elements of the array are formed by the application of a DNA solution to the surface of the array, the process is relatively slow. The development of VLSIPS™ technology has provided methods for making very large arrays of oligonucleotide probes in very small arrays. See U.S. Pat. No. 5,143,854 and PCT patent publication Nos. WO 90/15070 and 92/10092.

U.S. Pat. No. 6,284,460 describes methods for making arrays of oligonucleotide probes that can be used to provide the complete sequence of a target nucleic acid and to detect the presence of a nucleic acid containing a specific nucleotide sequence. One drawback to this method is that it relies on a new DNA synthesis chemistry as opposed to the standard phosphoramidite chemistry used in commercial DNA synthesizers. The technology feeds off the methods evolved in the electronics industry and therefore has some of the same requirements, such as, accurate positioning to micron scales, clean room requirements and the use of multiple photo-masks to define the array pattern. Although electronic “chips” (for example an Intel Pentium®™ microprocessor) are mass-produced economically, they are typically too expensive to be used as a disposable element, as is needed with a DNA chip.

Biochemical molecules on microarrays have been synthesized directly at or on a particular cell on the microarray, or preformed molecules have been attached to particular cells of the microarray by chemical coupling, adsorption or other means. The number of different cells and therefore the number of different biochemical molecules being tested simultaneously on one or more microarrays can range into the thousands. Commercial microarray plate readers typically measure fluorescence in each cell and can provide data on thousands of reactions simultaneously thereby saving time and labor. A representative example of the dozens of patents in this field is U.S. Pat. No. 5,545,531.

Currently two dimensional arrays of macromolecules are made either by depositing small aliquots on flat surfaces under conditions that allow the macromolecules to bind or be bound to the surface, or the macromolecules may be synthesized on the surface using light-activated or other reactions. Previous methods also include using printing techniques to produce such arrays. Some methods for producing arrays have been described in “Gene-Expression Micro-Arrays: A New Tool for Genomics”, Shalon, D., in Functional Genomics; Drug Discovery from Gene to Screen, IBC Library Series, Gilbert, S. R. & Savage, L. M., eds., International Business Communications, Inc., Southboro, Mass., 1997, pp 2.3.1.-2.3.8; “DNA Probe Arrays: Accessing Genetic Diversity”, Lipshutz, R. J., in Functional Genomics; Drug Discovery from Gene to Screen, IBC Library Series, Gilbert, S. R. & Savage, L. M., eds., International Business Communications, Inc., Southboro, Mass., 1997, pp 2.4.1.-2.4.16; “Applications of High-Throughput Cloning of Secreted Proteins and High-Density Oligonucleotide Arrays to Functional Genomics”, Langer-Safer, P. R., in Functional Genomics; Jordan, B. R., “Large-scale expression measurement by hybridization methods: from high-densities to “DNA chips””, J. Biochem. (Tokyo) 124: 251-8, 1998; Hacia, J. G., Brody, L. C. & Collins, F. S., “Applications of DNA chips for genomic analysis”, Mol. Psychiatry 3: 483-92, 1998; and Southern, E. M., “DNA chips: Analyzing sequence by hybridization to oligonucleotides on a large scale”, Trends in Genetics 12: 110- 5, 1996.

Unfortunately, all of the array fabrication methods mentioned above suffer from the same general problem in that each element of each array is a unique synthesis or an application step. This is true even when array elements or entire arrays are simply duplicated or produced “in parallel”, or more accurately, concurrently. Since each element is a unique synthesis or application, there is a chance for variation between corresponding elements on different arrays or, for that matter, duplicated elements on the same array. Even in a photolithographic process, increasing the number of chips on a wafer (the substrate on which multiple arrays are produced) results in an increase in surface area, which increases demand on the chemicals used in photochemistry (assuming no change in chip size).

What are needed in the art are methods to enhance the amount of material that attaches to an adaptable solid support and means to increase the flexibility and reproducibility with which materials are applied to such a solid support. The present invention helps meet those needs.

### SUMMARY OF INVENTION

The instant invention relates to a method for producing devices comprising a plurality of adjacent cast channels within a block or mold, where each channel may comprise a different adhered, adsorbed or linked chemical or biological agent of interest. Further, such devices afford keeping the channels in an intrinsically fixed addressable location with respect to other channels. Optionally, such channels allow for checking that all elements of the channel maintain a constant arrangement, register or pattern throughout the length of the device after channel formation. Moreover, these channels allow for facile sectioning of the devices to produce large numbers of identical arrays or chips and for performing a variety of different binding reactions and quantitative biochemical analyses on individual arrays or chips to include, for example, enzymatic activities, immunochemical activities, nucleic acid hybridization, protein complexes and large or small molecule binding under conditions yielding, for example, fluorescence, optical absorbance or chemiluminescence signals. Such signals can be used, for example, to acquire images of the signals that can be processed electronically and compared to produce clinically and experimentally useful data.

The present invention envisages applying solid surfaces, such as rods or tubules, to moldable surfaces which become substantially fixed around such solids thereby allowing the moldable surface to conform to the contours of the applied solid. When the solids are removed, casting results, forming a channeled surface within the substantially fixed moldable surface.

The present invention also envisages deposition of the compounds or biological materials by using a matrix comprising the compounds or biological materials where the matrix is purged away from the compounds or biological materials or otherwise reversibly attached so that the compounds or biological materials are free to bind to the inner surface of the cast channels within a block or mold.

In another aspect, the compounds or biological materials can be added to pre-cast channels, wherein the removal of a casting matrix (material) presents resulting surfaces that allow adsorption, adhesion or linkage of compounds or biological materials to occur within the cast channels. Further, means are disclosed to aid removal of such casting matrices (materials or solids) including, but not limited to, centrifugation, application of negative or positive pressure, countersinking ends, exploiting coefficients of expansion, heating, cooling, burning, drilling, etching, dissolving, coring and pulling.

The invention also relates to a method of generating surfaces comprising bio-reactive components such that the components are uniformly distributed within a defined area at a high density at a reactive surface. In a related aspect, methods are envisaged comprising placing solid containing compounds or components in a solidifying matrix onto a moldable surface, removing the matrix and retaining the entrapped compounds or components on the inner surface of channels formed in the moldable surface by adhesion, adsorption or linkage thereto. Moreover, retaining the reactive compounds may comprise purgation of the matrix, where optionally the purgation step uniformly deposits the entrapped bio-reactive samples on the available molded surface.

In a related aspect, the agents or compounds are deposited on the inner surface of such channels after removal of the casting matrix where such removal presents moieties for the adhesion, adsorption or linkage of deposited agents or to the moieties are separately generated after removal. Further, such agents or compounds are added subsequent to matrix removal. In a related aspect, the inner surface may be derivatized subsequent to matrix removal and prior to compound or agent deposition.

In a further aspect, the bio-reactive samples are embedded or immobilized in a meltable/reversible/degradable/dissolvable or otherwise removable matrix, which may comprise rods or tubules. Each rod or tubule may contain different or identical entrapped material samples. Alternatively, the rods or tubules are devoid of bio-reactive sample and functions solely for casting channels within a suitable mold or block. Further, the rods or tubules can be coated to allow for removal or for deposition of functional groups on channels formed therefrom. Still further, the rods or tubules can be used for checking that all elements of the mold or block maintain a constant arrangement or pattern throughout the length of the mold or block prior to matrix removal. Moreover, rods or tubules function to cast channels for subsequent sectioning of molds or blocks to produce large numbers of identical chips for forming desired patterns (arrays) on solid surfaces. Moreover, the resulting arrays are used for performing a variety of different quantitative biochemical analyses based on enzymatic activities, immunochemical activities, nucleic acid hybridization and small and large molecule and complex binding. These analyses are performed under conditions yielding to detection by, for example, fluorescence, optical absorbance, electrical or chemiluminescence signals, where these signals that are electronically processed and compared to produce clinically and experimentally useful data. The components can include, but are not limited to biological macromolecules, complexes, organelles, biological cells (i.e., prokaryotic and eukaryotic) and viruses. For example, the macromolecules can include, but are not limited to proteins, carbohydrates, nucleic acids and lipids.

In another aspect, the matrix (solid tubules or rods) can be made from various solid materials including, but not limited to super-cooled liquids, crystals, crystal polymers, non-crystal polymers, gels, waxes, emulsions, highly thickened or very viscous liquids, metals, low melting point alloys, colloid suspensions and cleavable linkages to a solid. In some situations, for example, the matrix may be as simple as ice or other material with a higher or lower melting point than the surrounding material.

In another respect, the moldable surfaces may be made from various materials, including but not limited to, suitable polymers, copolymers, their blends and reactive prepolymers. Examples of these materials include monomer or pre-monomer plastic, hydroxyethylmethacrylate (HEMA), polyvinyl alcohol (PVA), methyl metacrylate and polymethyl methacrylate (PMMA), copolymers of methylmethacrylate, polystyrene, reactive polyurethanes, etc. Other materials such as wax, gel, soft metal, glass, ceramic and other easy to work with materials including liquids which solidify upon cooling such as water freezing to ice.

In one aspect, the invention relates to devices that contain or are coated therein with agents of interest and methods for manufacture thereof.

The invention further relates to means and methods for constructing devices of the invention in which channels comprising a mold of any shape is used to form a channel device. In a related aspect, immobilized agents of interest are within or on the inner surface along the entire length of the channels comprising the molds.

The invention also relates to methods for constructing devices where the channels therein intrinsically form addressable locations in which the position of each channel relative to all others is retained throughout the block or mold comprising said channels. Upon sectioning, such a device would afford microarray products comprising two-dimensional surfaces that retain addressable channel locations by virtue of said construction.

In a related aspect, the invention relates to the preparation of microarrays wherein the molds or blocks are cut transversely many times at short intervals to yield cross sectional slices thereof to form microarrays and a microarray so prepared.

In another aspect, the instant invention relates to casting channels containing agents of interest, or means for immobilizing at least one or a class of agents of interest thereto. In a related aspect, the channels cast contain different immobilized agents of interest.

In an additional aspect, the invention relates to means for embedding or attaching whole or fragments of biological cells, tissues or infectious agents to channels in such a manner that the biologicals are exposed on the cut end of each channel.

The invention further relates to a method for the large scale production of identical flat two-dimensional arrays of immobilized nucleic acid-based agents for use in nucleic acid sequencing, in the analysis of complex mixtures of ribonucleic acids (RNAs) and deoxyribonucleic acids (DNAs), and in the detection and quantitation of other analytes including proteins, polysaccharides, organic polymers and low molecular mass analytes, by sectioning the cast channel device.

In a related aspect, the invention relates to exploiting microarrays for mass screening of large numbers of samples from one to a large number of agents of interest.

In a further related aspect, the invention relates to the development of sets of tests on different chips or microarrays done in optionally branching sequence, which reduces the cost, delay and inconvenience of diagnosing human diseases or other analytical purposes, while providing complex data ordinarily obtained by time-consuming sequential batteries of conventional tests.

In still another aspect, the invention relates to the fabrication of identical arrays that are sufficiently inexpensive to allow multiple identical arrays to be mounted on the same slide or test strip, and cross-compared for quality control purposes.

In a still further aspect, the invention relates to the incorporation of a non-fluorescent dye or other light absorbing material in the substance of the array (e.g., the moldable matrix) to control the depth to which light used to excite fluorescence penetrates the array, thereby controlling the depth to which fluorescence analytes are detected, and insuring that fluorescent analytes which diffuse too deeply into the content of the channels, and therefore do not diffuse out, are not detected.

In an additional aspect, the invention relates to the reproducible manufacture of biochips or microarrays for bioanalysis.

In a further aspect, the invention relates to the design and production of arrays, which are specifically designed to detect and diagnose a specific disease or condition.

In yet a further aspect, the invention relates to increasing the dynamic range of multiple-parallel assays by providing means for making serial measurements of fluorescence or absorbance over time, and for determining the rate of change of fluorescence or absorbance in each element of the array over time.

It is an additional aspect of the invention to produce biochips that are inexpensive and sufficiently standardized to allow more than one to be used for each analysis, and for controls and standards to be run routinely and simultaneously in parallel.

In a further aspect, the invention relates to the production of chips in which the array elements or channels may differ from one another in the composition of the immobilizing matrix or substrate, or the class of agent of interest may be different in different channels.

In an additional aspect, the invention relates to the production of chips in which different types of reactions may be carried out at each channel of the array, with the reactions including binding, immunological, enzymatic or hybridization reactions.

The invention further relates to the development of multiple parallel chip-based methods involving continuously increasing temperature such that temperature sensitive reactions may be carried out at physiological temperatures, followed by an increase in temperature to allow hybridization reactions to occur.

In another aspect, the invention relates to binding assays, where at least one analyte binds to at least one agent of interest. Further, said assay can be used to detect the presence or absence of analytes comprising test samples to include the use of labels to practice such assays. In a further related aspect, predetermined channels can be used to identify such analytes.

In a still further aspect, the invention relates to preparing libraries of compounds with each channel containing one of the compounds. The array may be used to screen simultaneously all of the compounds for a particular chemical or biological activity.

Other embodiments and advantages of the invention are set forth, in part, in the description that follows and, in part, will be obvious from this description and may be learned from the practice of the invention.

### BRIEF DESCRIPTION OF THE DRAWINGS

FIG. 1 depicts three rods of dissolvable solid material.

FIG. 2 depicts three rods aligned in a mold in a fixed parallel arrangement.

FIG. 3 depicts a matrix material being added to the mold to form a block with the rods embedded inside.

FIG. 4 depicts the effects of a solvent dissolving the rods and resulting in three parallel channels through the block.

FIG. 5 depicts the molded block with one splayed end for easy filling of the channels after rod removal.

### DETAILED DESCRIPTION OF THE INVENTION

The terms “binding component”, “binding partner”, “molecule of interest”, “agent of interest”, “ligand” or “receptor” and their grammatical variations, may be any of a large number of different molecules, biological cells or aggregates, and the terms are used interchangeably. Each binding component is immobilized at a channel of the array and binds to an analyte being detected. Therefore, the location of a channel containing a particular binding component determines what analyte will be bound. Proteins, polypeptides, peptides, nucleic acids (nucleotides, oligonucleotides and polynucleotides), antibodies, ligands, saccharides, polysaccharides, microorganisms such as bacteria, fungi and viruses, receptors, antibiotics, test compounds (particularly those produced by combinatorial chemistry), plant and animal cells, organelles or fractions of each and other biological entities may each be a binding component if immobilized on the chip. Each, in turn, also may be considered as analytes if same bind to a binding component on a chip. Non-natural specific binding components may also be used such as an aptamer, PNA, etc.

When a molecule of interest has a high molecular weight, it is referred to as a “macromolecule”. In terms of some biopolymers, high molecular weight refers to greater than 100 amino acids, nucleotides or sugar molecules.

The terms “bind” and grammatical variations thereof, include any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measured. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The terms “matrix” or “substrate” and grammatical variations thereof, mean an inert material which may serve as a solid phase.

The terms “channel” and grammatical variations thereof, mean a usually longitudinal passage, void or conduit. Pluralities, typically a large number, of channels are adjacent to each other in the form of an array within a moldable surface. Generally the channel can have any shape, such as having angular walls or arcuate walls. Generally the channel is enclosed. Thus, materials placed into a channel must be introduced from an end. A channel, and particularly a superficial channel in a block can be open, exposing said channel, and samples can be introduced into the channel through a longitudinal aperture. In such a case, if the channel contents are in a solid state, then the channel could remain unenclosed. However, an open channel can be made enclosed after adding channel contents by use of channel material, for example.

The term “cast,” including grammatical variations thereof, means to give a shape to a surface of a substance by allowing such a surface to form contours around a separate solid object, where the surface presented is subsequently fixed according to the outlines of the available contours of the solid object.

The terms “mold,” and grammatical variations thereof mean a cavity in which a substance is shaped. Typically, the mold will form a block having rectangular ends with the channels opening to the ends, however, the mold may form an amorphous shaped block which may be used as is or its exterior shaped to form a rectangular end.

The terms “remove” and grammatical variations thereof mean to cause evacuation of or to make free of something unwanted.

The terms “cells”, “sites”, “addresses” and “elements” herein refer to a unit component of an array identified by a unique address and these terms generally differ from other cells, sites or elements by their content as well as location. Biological cells are referred to by their type, e.g., microorganisms, animal and plant cells.

The terms “arrays” and “microarrays” are used interchangeably but may distinguish variations in the size of the cells. The instant invention involves the same methods for making and using either.

The terms “device” and grammatical variations thereof mean an article of manufacture resulting from the casting of solids in molds or blocks and their subsequent removal to produce a plurality of channels. In a related aspect, an array derived from the device (e.g., a microarray) would comprise a transverse section (perpendicular to the long axis of a channel) of the device, said section comprising a surface having an inert solid phase surrounding addressable reactive surfaces, where such surfaces contain agents of interest.

In a further related aspect, a positive reaction can be comprehended as a signal within the field (i.e., the whole reactive area or a sub-region therein can be visualized) or as a halo outlining the reactive area.

“Reactive surface(s)” or “RS,” and grammatical variations thereof mean the area on the microarray where the interaction between analytes and binding components occurs, where analytes and binding components include, but are not limited to, proteins, polypeptides, peptides, nucleic acids (nucleotides, oligonucleotides and polynucleotides), antibodies, ligands, saccharides, polysaccharides, microorganisms such as bacteria, fungi and viruses, receptors, antibiotics, test compounds (particularly those produced by combinatorial chemistry), plant and animal cells or organelles.

The term “uniformly distributed” refers to a substantially equal concentration of bio-reactive components within a defined analysis field (area). In a related aspect, for example, such a substantially equal concentration of bio-reactive components is considered “uniformly distributed” along the length of the channel.

Each device typically contains many channels (typically about 10 to a 100 or more) wherein each channel is at an intrinsically addressable location and contains a specific component of interest. Each array therefore contains numerous different components of interest.

The instant invention makes microarrays, “chips” or “biochips” by sectioning devices, where each channel contains an immobilized binding component, including biological molecules and entities such as nucleic acid fragments, nucleotides, antigens, antibodies, proteins, peptides, carbohydrates, ligands, receptors, drug targets, biological cells or subfractions thereof (e.g., ground-up cells, organelles, solvent extract etc.), infectious agents or subfractions thereof, drugs, toxic agents, natural products or extracts. In a preferred embodiment, a casting solid is embedded in a moldable surface, where subsequent purgation (i.e., solid removal) of the solid forms a channel in said moldable surface. In one embodiment, the channels are sealed at one end prior to casting. In other related embodiments, the channel can be modified consequent or subsequent to purgation.

The moldable surface may be of material such as glass, metal, ceramic or plastic. The binding components, e.g., nucleic acids, proteins, cells etc., may be attached, adhered or linked to the channel surface. Each device section cut constitutes a microarray for use in various binding assays. This binding can be done by any means known in the art such as, but not limited to, the methods of Burzio et al. (U.S. Pat. No. 5,817,470), Löfåas et al. (U.S. Pat. No. 5,716,854), Thust et al. (U.S. Pat. No. 5,955,335) and Hiriade et al. (U.S. Pat. No. 5,736,099).

A key aspect of the invention, which provides an economic advantage, is that the devices are prepared using only methods providing a functionality stable to long term storage are used. Unlike other methods involving protein containing liquids which must be prepared fresh each time, immobilized proteins in relatively dry form remain stable for great lengths of time, often without refrigeration.

For conventional products, quality control checks can sample only a small portion of a microarray population, which is unlike the instant invention where each device may be tested. Both the spotting technique and the in situ synthesis technique do not permit testing before completion.

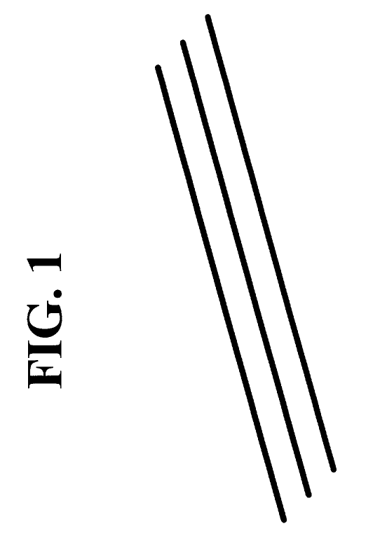

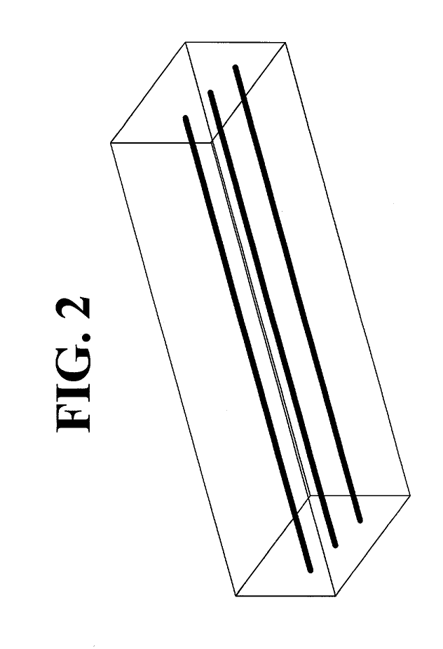

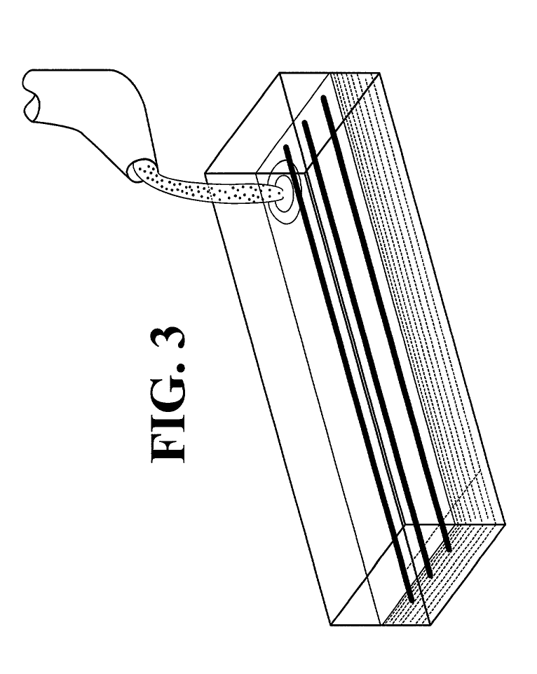

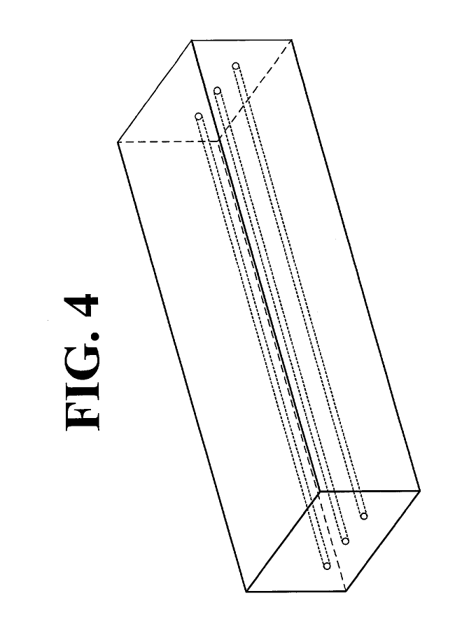

Various aspects of the invention and one simple format for making the present invention are illustrated in FIGS. 1-4.

“Meltable,” “removable,” “degradable,” “reversible” and “dissolvable” with regard to the casting solid are interchangeable terms referring to any medium that can: 1) be converted to a state, including, but not limited to a fluid and/or gas, that allows for facile partitioning away of said medium from another surface and/or 2) be cleaved, such as, separated from and removed from a solid phase to yield a void such as the inside walls of the moldable surface. An example of a meltable solid are low melting point waxes such as castor wax, stearyl alcohol, many polymers etc. An example of a removable solid is a silica. An alternative is any other particulate thickened medium, such as clays. An example of a degradable solid is a protease digestible gelatin or amylase digestible starch or a pH, chemical or temperature sensitive solid. An example of a reversible solid is alginate with calcium or sodium ions determined by the presence of a chelating agent and the like. An example of a soluble solid is a sugar or soap with water as solvent, or other lipophilic materials with organic solvents. The list of possibilities is very long.

In one embodiment, a reactive surface of a microarray is formed by coating a solid that forms the cast with a protein reactive composition. The solid is then embedded in a moldable surface to form the contours of the channel. As the solid is subsequently removed the inner surface of the channel is derivatized with protein reactive coating. For example, a glass fiber can be coated with (but not limited to) a protein reactive polymer (PRP, e.g., U.S. Pat. No. 4,543,211) and the PRP is allowed to dry/polymerize on the glass surface. The coated glass is then embedded in PMMA (polymethyl metacrylate), where the latter is allowed to form to the contours of said coated glass. The glass is then removed by treatment with ammonium fluoride and/or hydrofluoric acid (HF), and the resulting surface is thus derivatized to react with agents of interest.

In another embodiment, a glass or other solid fiber can be coated with, but not limited to, PMMA and allowed to dry or polymerize on the glass surface. The coated glass is then embedded in MMA (methyl methacrylate) or the prepolymer, where the latter is allowed to polymerize around the contours of said coated glass. The glass is then removed by treatment with HF and the resulting surface is thus derivatized to react with agents of interest.

In a related aspect, blocks formed in PMMA with channels formed by dissolving glass fiber can be treated with a solution of polyglycidyl methacrylate and a small amount of crosslinker. The reagent is removed by any various mechanical means, to include but not be limited to, positive/negative pressure or centrifugation, resulting in the etching of the channels. Such etched channels of PMMA will now have exposed epoxy groups available for reaction with agents of interest. Other materials and coating compounds with reactive moieties may be made and used as well.

In another related aspect, countersinking openings for easy addition of fluids subsequent to casting fiber or rod removal is also envisaged. This can be accomplished by using a casting fiber that has a thicker end or a flared out end at one pole. In one embodiment, countersinking is envisaged to occur at every other channel on opposing surfaces. The countersinking may be formed by physical means such as by drilling. In an another embodiment, non-countersinked openings could be plugged or casting would be such that the casting rod or fiber is not allowed to broach both sides of opposing surfaces.

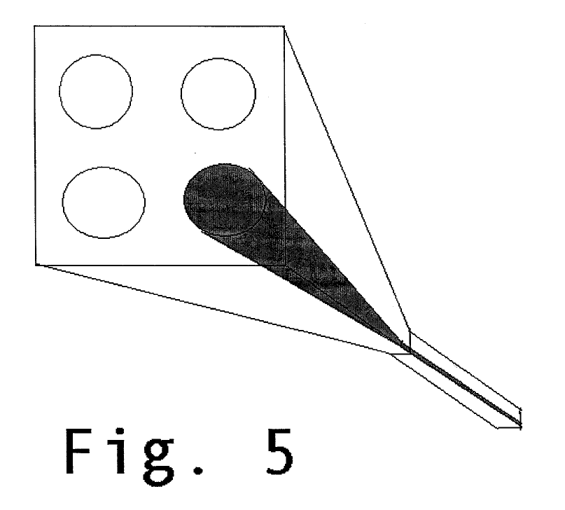

In one embodiment, splayed glass fibers are placed in a mold or block for easy filling of holes after dissolving. See FIG. 5. Alternatively, two different fibers or rods per channel may be used, a thinner one in the parallel region and a thicker or gradually expanding one in the spayed region. The tips of the two touching each other to create one channel once removed. Each may be removed by the same conditions or separately. In a related aspect, glass capillaries are used instead of solid rods and HF is pumped through the capillaries to enhance dissolving of glass. Likewise, other degradable materials may be used other than glass with a corresponding dissolving treatment or other melting, degrading, pulling treatment or sublimation (e.g., including but not limited to, naphthlalene, paradichlorobenzene, solid iodine, ice in a vacuum (i.e., lyophilization) and others can be found in CRC Handbook of Chemistry and Physics, 61^{st} ed. (Weast et al. eds.), 1980, at pp C-677 to C-678, CRC Press, Boca Raton, Fla.).

In another embodiment, metal wires or rods are used as casting materials for embedding in a mold or block. The mold or block is first heated, the metal wire is subsequently embedded and the mold is allowed to cool. Once cooled, the wires are pulled out and a channel results. The wires can be warmed to facilitate removal or extraction from the block, such as by passing an electrical current through the wire. In a related aspect, polymerization of the mold or block can be carried out at high temperature and cooled in a refrigerator, where the differences in coefficients of expansion may ease the removal of the metal wire. Alternatively, the metal wire may be coated with a lubricant before casting in the molds, for easy removal from the block, or with a composition one wishes to deposit on the inside of the channel.

In one embodiment, the metal may be made from solder or low melting point metal (e.g., wood's metal or gallium) and placed in an embedding block. After polymerization of the plastic, the metal is heated to melting temperature and using centrifugation or positive/negative pressure, the melted metal is removed. In a related aspect, this can be accomplished with two incompatible plastic materials, including but not limited to, polystyrene casting fibers and PMMA block or nylon casting fibers in a polystyrene block. In another embodiment, polysulfone casting fibers embedded in polytrifluoroethyl methacrylate or a PMMA is envisaged, where the fibers are dissolved in N-methyl pyrrolidone.

Reversible materials envisaged for use in the present invention can be comprised of, but not limited to heat reversible agar or agarose systems, reversible polyacrylamides, metal ion dependent alginate or other polysaccharide systems, redox dependent disulfide containing polymeric systems (e.g., polymers formed by oxidation of sulfhydryl groups to disulfides that can be reduced back to free sulfhydryl groups), and the like.

The degradation process can consist of, but would not be limited to, acid or base hydrolysis, enzymatic hydrolysis, photodegradation, temperature change such as with thermal responsive polymers which are solid or liquid, depending on the temperature, and other processes known in the art.

In a related aspect, the casting fibers are made of photosensitive/photoetching materials used in photolithography and the block or mold is UV transparent. By using UV light, the photoreactive casting fibers can be degraded or made amenable to dissolution by an appropriate solvent. The casting material is washed away leaving a block or mold with thin channels therein. In a further aspect, non-transparent blocks/molds with exposed ends may be used with one or several light-solvent-removal applications. Alternatively, a solid block of material that is a photosensitive/photoetching material is made where an opaque mask comprising holes is placed on a light exposed surface. Only the material not shadowed (behind the holes) will be degraded.

In another embodiment, channels may be formed mechanically by drilling holes into blocks. Such holes can be between from about 0.05 to about 1.0 mm, preferably about 0.1 to about 0.5 mm. Further, such mechanical means can include but are not limited to reaming or coring out a channel from a suitable solid phase (i.e., block or mold). In a related aspect, channels may be formed by laser ablation or a hot wire to burn holes in a suitable solid phase (i.e., block or mold).

Some channel surfaces may be used directly to immobilize reactants; others must be modified to allow such additions. Antibodies as will many other proteins will adhere to clean polystyrene surfaces, (Van Oss, C. J., & Singer, J. M. The binding of immunoglobulins and other proteins by polystyrene latex particles. J. Reticuloendothelial Society 3: 29040, 1966.) Polystyrene, in the form of microtiter plates, has been modified to bind nucleic acids, proteins and polysaccharides using techniques that are well known. Teflon® surfaces will tenaciously bind proteins or other macromolecules that have been suitably fluorinated (U.S. Pat. No. 5,270,193), and will bind fluorinated surfactants, which may render the surface hydrophilic, or positively or negatively charged. Glass, including controlled pore glass, may be modified to allow covalent attachment of antibodies, antigens or nucleic acids. Plastic surfaces may be modified non-specifically using corona plasma discharge or electron beam radiation and may then be coated with a variety of coatings or adhesives to which macromolecules may be attached. More specific covalent attachment of proteins, nucleic acids or carbohydrates may be achieved by a variety of modifications which attach reactive groups to polystyrene or acrylic surfaces, which groups, with or without extending linkers, will then couple under biologically compatible conditions to the biopolymers. Non-specific or specific coatings may be formed by chemical vapor deposition and other techniques. In addition to methods by which a receptor or molecule of interest is immobilized and used to bind an analyte, general methods exist for immobilizing members of a class of reactants. Binding may be accomplished by a number of affinity techniques such as protein A or protein G for antibody attachment, ligand/receptor pairs such as biotin-avidin, HIV-CD4, sugar-lectin or through a ligand that has a receptor such as digoxigenin-antidigoxigenin. For example, protein A or protein G may be immobilized and used to subsequently bind specific immunoglobulins which in turn will bind specific analytes. A more general approach is built around the strong and specific reaction between other ligands and receptors such as avidin and biotin. Avidin may be immobilized on a solid support and used to bind antibodies or other reactants to which biotin has been covalently linked. This allows the production of surfaces to which a very wide variety of reactants can be readily and quickly attached (see Savage et al., Avidin-Biotin Chemistry: A Handbook. Pierce Chemical Company, 1992).

Instead of attaching the binding partner to the inside wall of the channel, one may immobilize the binding partner in a material placed inside the channel. For example, solidifiable liquids containing the binding component may be pumped or drawn into a channel and allowed or made to solidify. A large number of polymerizing and gelling materials known per se may be used to entrap or covalently couple the binding component. These solidifying materials may chemically bind to the inside channels or be otherwise adhered.

In a related aspect, agents of interest can be immobilized on particles and subsequently added, as a slurry, to the inside of a channel. In one embodiment, said particles are mixed with a solidifying liquid and such a mixture is added to the channels.

A wide variety of methods have been developed to detect reactions between immobilized molecules of interest and soluble reactants. These differ chiefly in the mechanism employed to produce a signal, and in the number of different reagents that must be sandwiched together directly or indirectly to produce that signal. These include fluorescence (including delayed fluorescence) with the fluorescent tag covalently attached to the analyte, fluorescence involving soluble dyes, which bind to an analyte, and similar dyes whose fluorescence greatly increases after binding an analyte. The latter are chiefly used to detect nucleic acids. In more complex systems, including so-called sandwich assays, the result is the immobilization in the detection complex of a label such as a fluorescent label or an enzyme that, in combination with a soluble substrate, produces a preferably insoluble dye that may be fluorescent. Alternatively, the detection complex attached to the bound analyte may include a dendritic molecule, including branching DNA, to which are attached many fluorescent dye molecules.

In addition, protein or carbohydrate antigens may be detected using immunological reagents. Detection is generally by incorporation of a fluorescent dye into the analyte or into the second layer of a sandwich assay, or by coupling an enzyme to an analyte or a second or third layer of a sandwich assay that produces an insoluble dye, which may be fluorescent.

Microtomes for sectioning tissue blocks which may contain samples ranging from soft tissues to bone, often in blocks of embedding material (e.g., wax), are commercially available, as are a variety of techniques and arrangements for attaching sections to glass or plastic slides, for treating them automatically to remove some or all of the embedding media, and for systematically exposing the slides to a series of reagents.

Microtomes and other sectioning or cutting instruments capable of cutting assembled bundles of tubes, for example, into thin sections, and of maintaining their orientation after sectioning are known. In general, blade cutting is preferred to sawing to reduce contamination of binding components between cells of the microarray. Further, means of distributing and aligning separate chips may also be used. For example, after the fibers in a bundle may be fused or otherwise adhered to each other in a fixed pattern and the bundle is cut transversely or at an angle into many thin disks and portions are optionally dissolved if desired.

The sections (as microarray chips) may be attached directly to adhesive surfaces on flexible films or on solid surfaces, such as glass slides. In one embodiment, the entire block may be used as an assay device. After a reaction sequence (e.g., a binding assay) is complete, a thin section is cleaved from the block to remove the reacted surface and to expose a nascent microarray surface.

It is preferable that the solid phase be coated or derivatized to include moieties for specific or non-specific binding to the chip's inert solid phase. It is also feasible to attach sections (the word “section” is used here in place of “chip”) at intervals along a filmstrip, with others interleaved between them. Thus, a set of about a dozen or more sections that are different may be placed in repeating order along the film, and the film then cut in between to give one set of chips. For sequencing studies, one DNA insert may be amplified, labeled, and its hybridization to a large set of sections examined.

By using a non-deformable device, one can cut or saw the plurality of channels transversely thereby forming a large number of identical plates that are perfectly realignable. This permits highly consistent and reproducible arrays. By using an easily detectable material for channel identification, as a means for registering the microarray alignment, realignment is even easier.

A large number of different immobilization techniques have been used and are well known in the fields of solid phase immunoassays, nucleic acid hybridization assays and immobilized enzymes. See, e.g., Hermanson, Greg, T. Bioconjugate Techniques. Academic Press, New York. 1995, 785 pp; Hermanson, G. T., Mallia, A. K. & Smith, P. K. Immobilized Affinity Ligand Techniques. Academic Press, New York, 1992, 454 pp; and Avidin-Biotin Chemistry: A Handbook. D. Savage, G. Mattson, S. Desai, G. Nielander, S. Morgansen & E. Conklin, Pierce Chemical Company, Rockford Ill., 1992, 467 pp.

Most immunochemical or competition assays depend on a signal produced by a reagent other than the analyte. However, methods for fluorescently labeling all proteins containing aliphatic amino groups in a complex mixture reproducibly and quantitatively have been developed. Of these, CyDyes supplied by Amersham Life Sciences, and particularly, Cy2, Cy3 and Cy5 have proven most useful. When the components of such labeled mixtures are reacted with an array of immobilized antibodies, each specific antibody binds to one of the fluorescently labeled analytes, and the presence of each of the specifically bound labeled analyte can be detected by fluorescence. This method can be further improved by exposing the bound antibody array to a solution containing known subsaturating quantities of each analyte protein in a non-fluorescent form, washing the array, and exposing it to a test mixture of labeled proteins, thus producing a multiple competition assay.

Any of the conventional binding assay formats and detection formats involving an immobilized binding partner, known per se in hundreds of patents, may be used with the microarray systems of the present invention. Briefly, the microarray may have either plural ligands or plural receptors and the analyte may be either plural ligands or plural receptors. Competing elements that bind to either the analytes or the microarray cells may be added. The sample may be labeled and/or the competing element may be labeled and/or the microarray cell may be labeled. The labels may be interacting with each other to make a detectable signal or product or to quench a signal or product. The number of different combinations is in the dozens and any of them may be used in the present invention as well as different combinations for different cells of the microarray assay.

It is well known that several different clinical tests are often required to define a particular disease. These are often done serially, with one test or member of a battery of tests suggesting another, which in turn suggests a third test or group of tests, some of which are rarely done in local laboratories. There is therefore a need for inexpensive chips for the performance of a series of branching batteries of tests all at one time, using methods that produce accurate numerical results in a machine readable form, which are stable over time, and which are read by devices that can be compact and inexpensive relative to currently employed clinical analytical systems.

Many biochemical analyses require that the analytical procedure have wide dynamic range. Thus, enzyme and immunochemical assays are often done by determining the course of a reaction over a period of time, or by doing the analyses on a series of dilutions. Such analyses may be done by “reading” the microarrays at intervals during exposure to an analyte mixture of a developing reagent. This allows one to use an excess of labeled reagent that will initially detect high abundance ligands followed by overexposure of these microarray addresses and the beginning of detection of low abundance ligands. This system is most useful when the detection system involves an enzyme or slow reaction that becomes more intense over time such as the silver staining procedure used in photography and protein detection. In addition, parallel analyses using standards and blanks (controls) are required and are universally included. Large numbers of standardized inexpensive biochips will be required to meet these needs. These biochips may incorporate reactants of different classes that can, for example, detect and measure antigens, drugs, nucleic acids or other analytes.

Arrays have numerous uses other than determining bioactive properties. Chemical interactions and reactions may be tested as well, such as an array of different reactive chemicals being tested against a test substance or material to determine corrosion, electrochemical reaction or other interaction. This is particularly advantageous in the chemical formulations of plural substances such as in cosmetics, paints, lubricants etc. Alternatively, one may assay for desirable interactions between the analyte and all of the molecules of interest in the array.

The resulting surface may also be used for affinity chromatography, affinity separations, protein-protein binding to form protein complexes and the measurement for all of these.

The present invention also may coat the hollow channel surface with materials other than organic chemicals and biological materials. Different metals, anticorrosive coatings, decorative or instructional coatings, coatings for surface plasmon resonance (see U.S. Pat. No. 5,955,729), coatings for SELDI (see U.S. Pat. No. 6,020,208), combinatorial libraries of chemicals, and even coatings for depositing photosensitive and photoresists, electrically conductive coatings etc. such as are used in electronic integrated circuits.

By using the present invention, one avoids the difficulties of individually spotting each cell on a solid phase or forming a compound at each cell. This technique is limited by the spill, maximum practical concentration and ability to quantitatively measure small quantities of liquid. The technique using photosynthetic processes for making arrays is limited by the types of different compounds that can be synthesized on the solid phase. Both prior art techniques are expensive and require elaborate automated equipment or tedious labor as each array is individually produced. By contrast, the present invention is technically simple and quick where the “batch” can be in the thousands and much greater numbers of microarrays. The only individual effort required for each microarray are the steps of cutting and placement of sliced sections.

Microarrays prepared from sets of stored reagents or by the synthesis of different reactive sequences or compounds on the base chip present difficult problems in quality control. With large arrays, each reagent in its final form cannot be separately assayed in solid form before being used, nor can the correctness of the in-situ synthesized sequences be assured until after a set of arrays have been manufactured. If errors or substandard components are discovered in a batch of arrays, all must be discarded. These problems limit the use of “biochips” in routine clinical studies. It is well known that immobilized proteins and nucleic acids are more stable in a dry state than when in solution.

Large numbers of different and potentially new active compounds may be simultaneously screened by immobilizing them on individual channel surfaces, sectioning and forming a microarray. Peak fractions from separations, such as plant extracts may be simultaneously collected and used to form a microarray. The microarrays may then be used in a large number of assay systems simultaneously, dramatically reducing the time and effort to screen all of the compounds present for whatever activity one chooses.

Particularly preferred are large numbers of proteins or peptides and other combinatorial compounds generated by mass techniques. Different fractions from a separation technique from a natural source provide a resource of many different compounds and biological materials. A number of fractionation procedures are known to separate mixtures of many compounds. Different fractions or specific compositions may be used in the formation of a single derivatized channel. Two-dimensional electrophoresis gels from serum and other tissue and natural sources produce thousands of different proteins separated on the gel. Each may be individually removed (e.g., cut, eluted etc.) from the gel or separated by another method and used as the molecule of interest in the formation of a single channel. In such a method, with different devices being formed from different samples, protein differences between different samples may be readily compared.

When the immobilized macromolecules are antibodies, the microarray may be used to diagnose a variety of protein-based anomalies. A labeled second antibody to the protein(s) of interest may be used to further highlight the cell. In addition, the array may be used to immobilize infectious agents, which have been either previously stained or which, are stained after immobilization. Thus, microbes from biological samples, e.g., serum or plasma, may be concentrated, stained, for example, with a fluorescent nucleic acid stain such as TOTO-1 or YOPRO-1, and then allowed to find their matching antibodies on the array. They may then be detected by scanning for fluorescence and identified by position.

It is equally a part of the present invention to immobilize microorganisms or other molecules of interest and use them to localize antibodies from a patient's sera, discover the location of the latter using a fluorescent anti-human antibody, and thus, diagnose a disease which elicited antibody production in the first place.

Arrays have been prepared using phage display with inserts from specific genes, using synthetic oligonucleotides, or (to a limited extent) using displayed antigens or antibodies. In the present application, a population of peptide or antibody display phage may be used where each display phage is used to prepare a single derivatized channel. The molecule of interest may be bound to the surface of a channel per se. The phage, recombinant bacteria or other complex biostructure may also be fixed and the contained proteins cross-linked using glutaraldehyde or similar fixative, if desirable.

Each channel may contain a mixture of molecules of interest. For example, during chemical synthesis, a number of isomers are prepared. It may be convenient to not separate the isomers before forming a channel in some circumstances. Likewise, when fractionating a mixture, a channel comprising a mixture of receptors may be acceptable as total and complete isolation is difficult and time consuming, or such channels can be used for early screening.

Arrays may have an entire set of antigens/antibodies etc., in the various cells along with controls to effectively screen blood samples for common blood borne diseases before donated blood is provided for transfusion. Likewise, certain symptoms have a number of common causes that may be simultaneously screened for using arrays. For example, urinary tract infections are common and may be caused by a large number of different bacteria of varying sensitivity to various antibiotics. The simultaneous testing for a number of different factors would save considerable time and expense.

In the course of using a chip of the instant invention, various known techniques and materials are used to reduce non-specific reaction. Thus, in the case of a protein-based assay, the non-specific sites on the chip contributed by the substance of the mold or block, and essentially everything aside from the binding component of interest, are reacted with a blocking agent, such as albumin or milk, so that the blocking agent will bind to those areas not containing the binding component which could react with a ligand, analyte, reporter molecule or whatever would bind to the binding component, as known in the art.

Arrays may have cells that provide different concentrations of the binding component for quantitative measurement of an analyte. These provide internal standards for the microarray for both qualitative detection and quantitative detection. For example, a series of cells may contain different concentrations of an antigen. When a sample antibody is contacted with the cells and allowed to incubate, the varying binding signals in those cells and the absence of binding signal in another cell lacking antigen can be manipulated to provide an approximate binding affinity. The same can be done for determining minimal bacteriocidal concentrations when stained with a vital dye such as trypan blue or fluorescein acetate. Since a microarray may contain thousands of separate locations, one can determine the binding affinity of numerous antibodies simultaneously. Quantitative determination of other biological activities with either ligand or receptor immobilized in the channel may be used also.

Each channel in the sectioned two-dimensional array would contain relatively large numbers of binding components, such as lipids, carbohydrates, cofactors, DNA, RNA or protein molecules.

Because each channel has the molecule of interest essentially in the same form, as it will appear in the microarray, one can perform a quality control check on the an array slice. This is particularly important when the microarray is used for diagnostic purposes. Sampling microarrays from a batch may be a quality control check but it does not actually check the microarrays being sold. By contrast, small slices of the microarrays themselves are being used in the present invention. Assaying of such a slice represents an actual test of every microarray that will comprise a slice of the channel comprising device.

By contrast, with solid phase in situ synthesis of a molecule of interest directly on each cell of the microarray, none of the actual compositions to be used containing molecules of interest is actually tested for after it is synthesized. Rather periodic spot checking is relied upon for quality assurance. In microarray manufacture by spotting liquid droplets on a solid phase, one may test the liquids as a quality control check. However, these are liquid samples and do not represent the quality of the dry molecules of interest immobilized on a slide. Therefore, the quality control check is not the same as the actual product being sold. Again, one lacks any quality assurance for the actual compositions in the cells of the microarrays being sold.

For quality control in the present invention, the channels may be individually assayed, assayed in small groups or assayed as part of the whole device. More than one block may be bundled together before slicing with each block containing only part of the final array. Furthermore, by testing one final microarray, one has effectively tested all of them, as the composition of the slice is the same as that of any sliced product from the channel device.

For clinical tests, regulatory approval of tests and systems and methods for making them is required. When chips are fabricated using photolithography and other technology derived from electronic chip making, the cost of individual chips is quite high, and the possibility of error when chips are individually made is very high. Since chips are individually made and used only once, quality control is difficult and there is no good way of proving that any given chip is satisfactory. The best that can be done is to test a large fraction of a batch at random. With the present invention, a very large number of sections can be made from one composite assembly, and adjacent sections inter-compared as well as those some distance apart. Statistical analyses will be able to predict the rate of errors that may occur. However, of even greater importance is the fact that since the sections can be made in large numbers and quite cheaply, it will be feasible to run duplicate analysis on clinical samples, and to run confirmatory analysis when important diagnostic results are obtained. As a result, the present invention, for example, makes feasible widespread and routine application of genetic analyses in the practice of medicine.

For general clinical use, it is important to have identifiers on the slide holding the chip, and identifiers may be integral with the chip itself. The mold or blocks as well as individual microarrays may have machine readable indicia such as a barcode printed along one border to provide identification and orientation. In addition, small concentrations of dyes, usually non-fluorescent, may be incorporated into the polymers from which selected molds or blocks are made such that they present a pattern, for example, of one or more numbers, or one or more letters for orientation or identification purposes. It is also useful to have a few cells or elements which do incorporate fluorescent dyes or other detectable compound or structure and which serve to calibrate the measurements. It is further feasible to introduce dyes into the contents of selected surfaces to additionally identify them. If channels in an array are out of alignment giving rise to the loss of one channel in one line, this can be readily observed because the entire pattern will show a misalignment.

The mold or block material used to present the channels in an intrinsically fixed addressable location may be inert and/or opaque, while the channels and preferably, their contents will conduct light along their length. As a final check on the orientation of array elements, one element at a time at one end of the mold or block may be illuminated, and the light detected and related to array position at the other at the other end.

The choice of dissolving or removing the casting matrix depends entirely on the composition of the casting matrix and the agent of interest to adhere to the channel surface. The selection is at least as broad as that of the casting matrices. It is important that this casting matrix not adversely affect the agent of interest.

An advantage of the present system is that very large numbers of arrays may be cut, and some fraction of them used for standardization. For example, if a bar 100 cm in length were constructed, and if the bar were cut at 100-micron intervals, then 10,000 arrays would be available. If the sections were 10 microns in thickness, then the number of arrays would be 100,000.

If the individual channels were 100 microns in diameter with 100 micron spacer, there could be about 50 channels per row, and 10,000 in a block of channels with a cross-sectional area of 1 cm square. The present invention is the first array to have such a large number of different cells per unit area on a microarray without the binding agent being covalently attached to the chip. It is preferred for the present invention to have at least 10, more preferably 20, 50, 100, 250, 500, 1,000, 5,000, 10,000, 20,000, 100,000 or more cells per square centimeter of array. These are much higher concentrations than depositable cells formed by microfluidics in commercial microarrays.

To greatly increase the number of cells per square centimeter beyond even these high numbers, one may prepare a large mold or block with relatively large channels and stretch or draw the mold or block. This is typically done with deformable solids optionally with application of heat etc. While this makes the individual channels thinner, it does not affect their basic composition or their orientation with respect to each other and cross-section geometry. This technique has the twin advantages of allowing one to make more microarrays and making them smaller. By using a plastic mold or block medium such as a low melting point wax, the results are deformable or ductile channels that may be drawn to very thin channels of less than 20 microns in diameter. The field of drawing thermoplastic materials is well known per se. Even if not truly drawable through a die, one can pull or extrude plastic materials between rollers to lengthen and reduce the diameter of the channels. With optional application of gentle heat or solvent vapors, one need only pull the ends of the mold or block to generate the same lengthening and reducing of cross-sectional area. Channels may be drawn to even thinner dimensions thereby permitting microarrays of in the millions of addressable locations per square centimeter of microarray.

In the situation where the binding partner is heat stable, such as an oligonucleotide, the heating and drawing may be performed after immobilization inside the channel. For heat labile binding partners, the immobilization is preferably performed after the block is drawn.

The known photochemical processes of Fodor et al., Nature 364:555-6 (1993); Hacia et al., Molecular Psychiatry 3:483-92 (1998); and Fodor et al., Science 251:767-773 (1991) prepare short peptides and oligonucleotides covalently bound to the supporting chip. The process of amino acid or nucleotide synthesis inherently limits the practical length. Synthesis of entire proteins or genes on chips is not practical. Additionally, the secondary, tertiary and quaternary structure of the proteins may be important. By contrast, the present invention permits analysis of such proteins or genes.

Not only can the chips of the present invention be used to identify infectious agents by identifying characteristic nucleic acid sequences, they can also be used for identifying intact bacteria, mycoplasma, yeast, nanobacteria and viruses using arrays of immobilized specific antibodies or other specific binding agents.

This system may be used for the identification of viruses or other infectious particles isolated by microbanding tubes, see, for example, WO99/46047. Thus microbes from biological samples, e.g., serum or plasma, may be concentrated, stained with a fluorescent nucleic acid stain such as TOTO-1 or YOPRO-1, and then allowed to find their matching antibodies on the array. They may then be detected by scanning for fluorescence and identified by position. It is equally a part of the present invention to immobilize microorganisms or other molecules of interest in the described chips, to use them to localize antibodies from a patient's serum, and to then discover the location of the latter using a fluorescent anti-human antibody, thus diagnosing the disease which elicited antibody production.

By using the present invention, one avoids the difficulties of individually depositing a different reagent on each cell on a solid phase or synthesizing a different compound at each cell. The former technique is limited by both the possibilities of spilling and mixing reagents and by limitations in the accuracy of measurement of small fluid volumes. Further, many proteins are not stable over a long period of time in solution. If arrays are prepared from multiple liquid reagents, these must all be assayed at intervals to ensure adequate stability. Further complicating the use of proteins in liquids is that different proteins degrade at different rates, which may reduce reproducability with microarrays not stabilized by immobilization and/or drying. The latter technique is limited by the types of different compounds that can be synthesized on a solid phase surface. Both prior art techniques are expensive and require elaborate automated equipment or tedious labor to produce each array individually. By contrast, the present invention for producing microarrays is technically simple and quick, and the batch size may be in or exceed the thousands.

Because the mold or block is maintained, additional channels may be added to the mold or block as needed before sectioning additional arrays. This allows one to detect and measure newly discovered emerging diseases, new proteins, genes or compounds without recreating a completely new mold or block.

This invention may be applied in an alternative fashion in which the molds or blocks are stored at user sites, and the arrays only sliced off as needed. This arrangement may be useful for research purposes where identical arrays are required over the long term, but only a few are required at any one time.

The invention also allows different immobilization technologies, different classes of immobilized agents of interest, different classes of analytes, and different types of detection methodologies to be employed on the same chip.

Since channels are reproducible between and among molds or blocks, the location of each channel or cell may be accurately determined by mechanical means. Reference markings on polished edges or other suitable locations can further identify each cell in the array. Current commercially available computer driven two-dimensional drives of sufficient accuracy enable visualization or testing of individual cells, or material may be added thereto or withdrawn therefrom.

Surface treatment with a material repellant to the fluid to be eventually located inside each cell reduces cross leakage between and among cells. For example, fluorinating (Teflonizing) or silylating agents repel water thereby generating sufficient surface tension to reduce cross leakage between and among cells of the microarray. Additionally, should the borders of each cell be somewhat uneven, smeared or touching another cell, the material out of place may be burned off by application of a laser beam between the individual addresses of the microarray.

After sections have been cut from a mold or block, they are generally bound to a solid backing to provide structural support and ease of handling. The solid backing is typically a sheet of plastic, glass or metal, for example, although other materials may be used. The attachment may be done, e.g., by a temporary or permanent adhesive or heat fusion. Pressure sensitive adhesives are particularly preferred.

Individual cells in the array may be detected or visualized by scanning the entire array or portions thereof (e.g., one or a few cells) with a charged coupled device (CCD) or by illuminating one or a few cells at a time, such as by a condenser lens and objective lens. The absorbance and emission of light may thus be detected. Detection may be based on a large number of detectable labels including radioactive, enzyme, luminescent, optically absorbent dye, magnetic, spin-labeled, oxidizers or reducers, chemiluminescence, electrical conductance or indirect labels which interact with a detectable component interacting with the agents of interest in the microarray. The preferred detectable labeling system is based on fluorescence, usually epifluorescence. This requires that the interrogating sample be labeled with one or more fluorescent dyes. The amount of test material required is very small since it would be applied to the arrays as a thin dilute film. Hybridization of nucleic acids would be done under conditions of carefully controlled stringency.

To distinguish selected channels, one may either seal off the selected channels and/or fill them with an easily detectable substance. Different colored inks, dyes and colored materials are particularly well suited as well as detectable components similar to or opposite from the detectable component(s) being detected in other cells. Printing methods with drying inks or plastics, sublimation, solvent containing an ink or ink-jet printing may be used. The indicia so formed permits better alignment or easily detectable marking when the array is in use. This permits easy optical alignment.

Once the microarray has been used in a binding assay and the ligands are bound to the receptors, in certain instances it may be useful to provide further identification of the ligand. In certain situations, one does not know the entire structure of the ligand from the receptor that specifically binds to it. For example, if the ligand is a cell, a macromolecular complex, or a derivatized molecule with the derivatized portion acting as the ligand etc., further analysis may be desirable. In this situation, one may elute the ligands from the microarray and collect them for further analysis. For antibody/antigen binding, a pH 2-3 environment or other conditions that disrupt binding of antigen to antibody should strip the ligands. For nucleic acid hybridization, raising the temperature should strip the ligands. A variety of other chemical, physical and electrical techniques for breaking such bonds are known per se.

To enhance specificity of the elution process, an electrode may be placed directly over one or a subset of cells and a current passed through the microarray to release the ligands at that location. The electrode may also be part of a micropipette system to collect the released analytes, see U.S. Pat. No. 5,434,049. Preferably, one uses a porous membrane and applies a current on opposite sides of the membrane. Thin wires may be applied on, in or behind the solid surface. The same or similar system may be used to assay for binding by using a label which alters electrical resistance.

The method used for analysis of the eluate may be capillary electrophoresis, mass spectrometry or a second binding assay. Convenient to mass spectrometry, the microarray itself may be introduced into a laser-desorption system incorporated into a mass spectrometry system wherein bound molecules are desorbed and analyzed.

Once the analytes have been striped from the microarray, the microarray may be reused. This reuse process has the advantage of being standardized by multiple controls over time.

The previous methodology for preparation of protein chips requires preparation, use and reuse of large numbers of proteins in solution. Proteins, nucleic acids, biological cells, other chemicals and complexes in solution are unstable and deteriorate over time. Even if frozen, repeated use may involve repeated freeze-thaw cycles that denature certain proteins as well. By contrast, immobilized proteins have been shown to be stable over long periods of time.

A preferred embodiment of the device of the present invention includes a casting phase and a second moldable phase. The casting phase may be in the form of, for example, polymer, particles of sugars or other water or solvent soluble materials, such as inorganic particles such as calcium carbonate particles, which can be dissolved in dilute acid. This includes any combination of a first solid phase that can be contoured using a second phase and upon removal forms a channel within the second phase. For example, a combination of a first solid casting phase of any material which is dissolvable in a solvent embedded in any second material not dissolvable in the same solvent may be used in the present invention.

Solid plastic casting materials also can be prepared which incorporate polystyrene latex or other plastic composition to which proteins or nucleic acids are attached. Conditions can be arranged such that the casting plastic is eroded so as to leave a film or residue on the inner surface of a formed channel within the mold or block (e.g., Teflon® coated inner channel surface). For example, proteins derivatized with fluorinated groups attach strongly to Teflon®. Such derivatized Teflon® in, for example, an acrylic plastic or other suitable casting plastic, can be partially removed from the inner molded surface by a dilute acrylic solvent, composed, for example, of methylene chloride and ethyl alcohol.

A variety of methods for derivatizing plastics and for attaching polypeptides, proteins, nucleic acids, polynucleotides, saccharides, polysaccharides and small molecules thereto have been developed and are known to those skilled in the arts.

Note that cast channels described herein can be produced with a casting material comprising string or thread through the center thereof to facilitate removal.

In another embodiment, arrays from the instant device may have the molds or blocks coated with biomolecules either covalently or in suitable polymer coatings. Isocyanate polymers, such as oxyethylene-based diols or polyols wherein most if not all of the hydroxyl groups thereof carry polyisocyanate groups, are suitable. Some such polymers can be comprised of polyurea/urethane polymers. The polymers are well hydrated and fall in the category of hydrogels. Suitable starting materials include triols, such as glycerol, trimethylpropane and triethanolamine, tetrols and polyethylene glycols. Suitable polyisocyanates include diisocyanates and such. The polyisocyanates can be aromatic, aliphatic or cycloaliphatic. (Braatz et al., U.S. Pat. No. 5,169,720 and Braatz, J. Biomaterials Applications 9:71-96 (1994)).

In another embodiment, the device includes molecules of interest attached to the inner surface of the channels. The section arrays may be considered as ultramicrotiter plates and may be used for flow-through analysis based on, for example, immobilized affinity ligand techniques (Hermanson et al., Immobilized Affinity Ligand Techniques, Academic Press, 1992, p 407), for polymerase chain reaction (PCR) amplification of immobilized oligonucleotides, or for other detection reactions and the like that can be accomplished at that scale, as described, for example, in U.S. Pat. No. 5,843,767. When the channels comprise Teflon® with the internal surfaces treated to become hydrophilic, the cut ends will remain hydrophobic. When a hydrophilic test solution is spread across the surface of the chip, the solution tends to flow into the channels in self-controlling volumetric amounts, and, if the total amount of fluid is controlled, properly tends not to affect adjacent cells. The upper and lower surfaces then can be sealed with a suitable adhesive tape and the whole subjected to reactions, for example, for polymerase chain reaction amplification of DNA. Alternatively, the sandwiched structure may employ two pieces of material such as glass or quartz to seal the ends of the channels, creating microchambers. Changes in fluorescence or in optical absorbance may be detected in each channel through the transparent end windows, and the reaction followed calorimetrically or fluorometrically. The channels, their filling material or the surrounding block may act as a light pipe to permit analysis of the signal through the respective materials themselves such as the channel (or its filling) acting as an optical fiber.

A variety of other reactions may be performed inside the microarray. For example, a polypeptide, polysaccharide or polynucleotide may be synthesized in situ and/or a library of combinatorial small molecules such as esters, amides, carboxylates etc., prepared. The same reactions, including PCR, may be performed in any of the other types of channels.

The inside surface of the channels described may be modified chemically to allow attachment of polynucleotides, polypeptides, polysaccharides or other molecules either directly or through linkers.

Similarly, if the channels contain candidate molecules for binding to a hormone receptor, the immobilizing and attaching method and means are those that retain the configuration of the candidate molecules to allow recognition and binding by the hormone receptor.

In addition, many protein or carbohydrate antigens may be detected using immunological reagents. Detection is generally by incorporation of a fluorescent dye into the analyte or into the second layer of a sandwich assay, or by coupling an enzyme to an analyte or a second or third layer of a sandwich assay that produces an insoluble dye, which may be fluorescent.

The art of using centrifugal force to fill short lengths of tubing with viscous media can be modified to fill or empty the channel of a channel containing block.

The microarrays can be of any thickness as required by the anticipated use thereof. Another determining factor might be the rigidity of the blocks. In a one embodiment, the sections will be less than 1 cm in thickness. In another embodiment, the sections will be less than 50 microns in thickness. As will be exemplified in further detail hereinbelow, sections can be on the order of microns in thickness.

When one wishes to enhance binding between analyte and binding partners on the reactive surface of the microarray, one may produce a channel surface that contains ridges or other structures to increase surface area of the walls of the channel (e.g., rifling, the act of making inner surface spiral grooves). In another embodiment, a casting fiber can be coated with a porous forming plastic (e.g., an open cell foam, see U.S. Pat. Nos. 5,506,035; 5,491,980; 5,485,976) and subsequently embedded in an impermeable material producing a thick spongy ring having a greater surface area for agent immobilization (e.g., higher signal density). The inside surfaces of the channel may be made porous or a porous material added to increase the surface area and thus provide for more binding sites for the binding partner. The method may simultaneously add reactive moieties to the surface. Alternatively, a three dimensional surface provides for better attachment for gels, polymers and other materials used to fill the channel and which immobilize the binding component. The following examples are included for purposes of illustrating certain aspects of the invention and should not be construed as limiting.

### EXAMPLE 1

### Formation of a Channel Block

16 steel wires, 0.5 mm diameter, were arranged in 1 cm ID UV transparent glass tube with Teflon stoppers at both ends. The stopper had holes in a regular pattern so that a 4×4 square arrangement of the steel wired is maintained. With one stopper off and the other holding the steel wires in place, the glass tube was filled with methyl methacrylate and the other stopper inserted such that the wires extended through it. The methyl methacrylate is polymerized by UV light and then the glass tube broken to remove the cast having steel wires embedded therein. The Teflon stoppers are also removed. The block is clamped and the steel wires are removed individually by pulling with pliers. The resulting block has 16 parallel channels in a fixed orientation.

### EXAMPLE 2

### Formation and Analysis of a Microarray

Antibodies are purified by affinity chromatography by reversible binding to the respective immobilized antigens.

Glass fibers are aligned in parallel fashion and embedded in a methyl methacrylate monomer or prepolymer block of PMMA. After polymerization, the glass fibers are dissolved by soaking the block in hydrofluoric acid. After dissolution, centrifugation to remove the dissolved glass and drying, the channels are treated with a solution of polyglycidyl methacrylate and a small amount of crosslinker (e.g., polycarboxylic acid). Subsequently, these reagents are removed by centrifugation, leaving epoxy groups exposed on the PMMA channel surfaces.

Antibodies directed against human serum albumin (HSA), transferrin (Tf) and haptoglobin (Hp) are used. A total of three antibody sera are used in tests with: 1) rabbit anti-HSA, 2) rabbit anti-human Tf and rabbit anti-human Hp and 3) mixed anti-HSA, Tf and Hp.

Aliquots of each purified sera are added to uniquely identifiable epoxy-group comprising channels and the antibodies are allowed to incubate within the channels under condition that maximize conjugation of the epoxy group with available functional groups on the proteins. Pre-immune rabbit serum is used as the control.

After incubation of the antibodies within the block, transverse sections are prepared by slicing thin slices perpendicular to the long axis of the channels, where the slices are then mounted on a glass slide. The sections reveal a pattern of 0.5 mm areas (reactive surfaces).

To test specific protein binding to the exposed surfaces of a section forming the microarray, commercially available HSA and Tf protein are labeled with fluorescein isothiocyanate (FITC) on Cellite (Sigma), a carrier for insoluble FITC. The proteins are dissolved in about 4 ml of 0.4 M sodium bicarbonate buffer (˜pH 8.3) and added to the dry FITC on Cellite in the following amounts:

| | |
|---|---|
| ˜4.5 mg HSA | 30 mg FITC on Cellite |
| ˜2.8 mg Tf | 18 mg FITC on Cellite |
| ˜4.5 mg Serum Protein (20 μl) | 10 mg FITC on Cellite |

The reaction is conducted at room temperature for 30 minutes. The Cellite is removed by centrifugation, and the supernatant protein and unreacted dye placed in a centrifugal protein concentrator, where the protein is washed by repeated dilution and re-concentration in buffer. The fluid is centrifuged to remove the Cellite and the supernatant recentrifuged with 4 ml sodium bicarbonate buffer until clear.

Sections of the array on a glass microscope slide are exposed to a solution of fluorescently labeled HSA. During the exposure of the section, the protein is expected to interact specifically with the antibodies present on two sites (round areas on the section): those bearing antibodies to HSA and the mixed anti-HSA, Tf and Hp. Labeled HSA does not interact with the sites carrying antibodies to Tf alone or to the site comprising exposure to pre-immune sera.

The sections are examined under an epifluorescence microscope equipped with a 500 nm low pass filter and a 510 nm high pass filter for fluorescein fluorescence detection and a 35 mm camera.

### EXAMPLE 3

### Formation and Analysis of a Microarray

The method of Example 2 is repeated using 0.1 mm thick nylon filaments imbedded in polystyrene. The nylon is dissolved in 70% acetic acid. The remainder of the procedure is the same as above. It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated herein by reference in their entirety.

What is claimed:

1. A device comprising a plurality of parallel channels adjacent to other channels within a suitably shaped block at addressable locations with respect to each other wherein said channels are formed by removing a material from within a solid block thereby forming said channels, and further wherein said channels contain different agents of interest by immobilizing said agents within separate channels.

2. The device according to claim 1, wherein said suitably shaped block comprises materials selected from the group consisting of polymers, copolymers, their blends, and polymers formed by reactive monomers, prepolymers and polymers.

3. The device according to claim 1, wherein the agent of interest is selected from the group consisting of a microorganism, ligand, antibody, antigen, nucleic acid, polysaccharide, protein, combinatorially produced compound, receptor, plant or animal cells, organelles and fractions thereof.

4. The device according to claim 1, wherein said agents are adhered, adsorbed or linked to the inner surface of each separate channel.

6. The device according to claim 1, wherein all or most of the channels contain a different immobilized agent of interest.

7. The device according to claim 1, wherein at least one of the channels contains a dye.

8. The device according to claim 1, wherein different channels contain different concentrations of the same agent of interest.

9. The device according to claim 1, wherein each channel contains no more than one immobilized agent of interest.

10. A method of forming a device comprising a plurality of parallel channels adjacent to other channels within a suitably shaped block at addressable locations with respect to each other wherein said channels are formed by removing a material to form said channels, and further wherein said channels contain different agents of interest by immobilizing said agents within each separate channel by means of said removal comprising:
(a) embedding a plurality of solid materials in a moldable surface of a blocking forming material;
(b) removing said solid materials embedded in a block to form the channels; and
(c) adhering, adsorbing or linking at least one agent of interest to the inside of the channel of step (b).

11. The method of claim 10, wherein said at least one solid material is a fiber or rod and further comprising aligning said fiber or rod with other fibers or rods in a parallel fashion before embedding.

12. The method of claim 11, wherein the solid material is selectively degradable under conditions where the block is not degraded. .

13. The method of claim 11, wherein the solid materials are removable from said block to form the channels.

14. The method of claim 11, wherein said solid material is a solid fiber or rod embedded in a suitably shaped block comprises materials selected from the group consisting of polymers, copolymers, their blends, and polymers formed by reactive monomers, prepolymers and polymers.

15. The method of claim 10, wherein removing step (b) further comprises:
(i) exposing of available moieties of the inner surface of said channel comprising said moldable surface or
(ii) derivatizing the inner surface of said channels comprising said moldable surface.

16. The method of claim 10, wherein said removing step is selected form the group consisting of degrading, dissolving, withdrawing, melting, sublimation and washing of the embedded solid.

17. The method of claim 10, wherein the means of said removal is selected from the group consisting of centrifugation, application of negative pressure, application of positive pressure, countersinking alternate polar ends, exploitation of coefficients of expansion, heating, cooling, burning, drilling, etching, coring and pulling.

18. The method of claim 10, wherein said moldable surface becomes substantially fixed around said solid material thereby allowing said moldable surface to conform to the contours of said solid, further wherein removal casts a channel surface within the substantially fixed moldable surface.

19. A method for making an array comprising forming the device of claim 1 and cutting the device transversely or at an angle to form a section such that a fixed position of the at least one channel with respect to a plurality of other channels is maintained.

20. The method of claim 19, wherein said sections are less than 1 mm thick.

21. An array prepared by the method of claim 19, comprising a plurality of channels in intrinsically addressable locations on the array, each channel containing an agent of interest immobilized within or on the inner surface of said channel, wherein different channels contain a different agent of interest immobilized therein or thereon, and wherein each agent of interest is located at a known address.

22. A binding assay for detecting an analyte in a sample wherein said analyte binds to at least one agent of interest in an array comprising;
(a) contacting a sample suspected of containing an analyte with the array of claim 21 under conditions permitting the binding of analyte to agent of interest;
(b) detecting the presence or absence of binding between analyte and each channel in the array; and
(c) determining the presence or absence of the analyte by the presence of any binding being detected at a predetermined channel of the array.

23. The binding assay of claim 22, further comprising;
(i) adding a labeled detection agent capable of binding to channels having either analyte bound to agent of interest or channels not having the analyte so bound, but not both, and
(ii) detecting the presence of the labeled detection agent in one or more channels of the array.

24. An array prepared by the method of claim 19, comprising at least about 50 reactive surfaces per square centimeter wherein each reactive surface contains an agent of interest that is not chemically bound to the inner surface of said reactive surface.

25. The microarray of claim 24, containing at least about 250 reactive surfaces per square centimeter.

26. An array prepared by the method of claim 24, comprising at least about 500 channels per square centimeter wherein each channel contains an agent of interest selected from the group consisting of a macromolecule, a complex, a microorganism, a plant or animal cell, an organelle or a fraction of a biological cell.

27. A method of forming a device comprising a plurality of parallel channels adjacent to other channels within a suitably shaped block at addressable locations with respect to each other wherein said channels are formed by removing solid material to form said channels, and further wherein said channels contain different agents of interest by immobilizing said agents within each separate channel by means of said removal comprising:
(a) removing said solid materials from said block to form the channels and
(b) adhering, adsorbing or linking at least one agent of interest to the inside of the channel of step (a).

28. The method of claim 27, wherein the solid material being removed is of the same material as the block.

29. The method of claim 27, wherein the solid material is removed by heating, cooling, burning, drilling, etching and coring.

30. The method of claim 27, wherein the block is made of materials selected from the group consisting of polymers, copolymers, their blends, and polymers formed by reactive monomers, prepolymers and polymers.
